(19) United States
(12) Patent Application Publication
(10) Pub. No.: US 2026/0232595 A1
(43) Pub. Date: Aug. 13, 2026

(54) Health Supplement Composition, and a Method of Manufacturing a Health Supplement Composition

(71) Applicant: Kim Jean Clarke-Black, Cape Town (ZA)

(72) Inventor: Kim Jean Clarke-Black, Cape Town (ZA)

(21) Appl. No.: 19/637,241

(22) Filed: Apr. 2, 2026

Related U.S. Application Data
(63) Continuation of application No. PCT/ZA2025/050013, filed on Mar. 19, 2025.

(30) Foreign Application Priority Data
Aug. 20, 2024 (ZA) 2024/06369
Oct. 18, 2024 (ZA) 2024/07900
Oct. 18, 2024 (ZA) 2024/07901

(51) Int. Cl. A61K 9/20; A61K 9/00; A61K 9/16; A61K 9/48; A61K 31/122; A61K 31/593; A61K 33/10

(52) U.S. Cl. CPC A61K 9/2018; A61K 9/0095; A61K 9/1623; A61K 9/1682; A61K 9/2095; A61K 9/4833; A61K 9/4858; A61K 31/122; A61K 31/593; A61K 33/10

(57) ABSTRACT

A health supplement composition comprises a therapeutically effective amount of at least one active ingredient selected from Calcium Carbonate, Maize Starch, Magnesium Stearate, Microcrystalline Cellulose, Vitamin D3, and Vitamin K2; and at least one pharmaceutically acceptable excipient selected from the group consisting of binders, disintegrants, stabilizers, lubricants, diluents, and preservatives, wherein the composition is formulated for oral administration in a powder, tablet, capsule, or liquid dosage form. In an embodiment, the composition is provided in a powder/granular form, typically packaged in tablets, as well as in a liquid form or as an additive to a liquid such as water, juice, or vegetable glycerine. Other delivery forms for the novel supplement may be utilized to meet particular operating circumstances or environments. Consequently, the composition is not limited to a specific delivery system but covers any and all suitable delivery systems.

### CROSS REFERENCE TO RELATED APPLICATIONS

This is a continuation of International Patent Application No. PCT/ZA2025/050013 filed on Mar. 19, 2025, which claims priority to South African Patent Application 2024/06369 filed Aug. 20, 2024, South African Patent Application 2024/07900 filed on Oct. 18, 2024, and South African Patent Application 2024/07901 filed on Oct. 18, 2024, which are hereby incorporated by reference in their entirety.

### TECHNICAL FIELD

The present disclosure relates to a health supplement composition, and a method of manufacturing a health supplement composition.

### BACKGROUND

Many health supplements available today suffer from poor bioavailability, suboptimal ingredient interactions, and inconsistent efficacy. Existing formulations, such as single-ingredient vitamin supplements, multi-vitamins, and prescription-based nutritional therapies, have limitations, including low absorption rates, potential ingredient interactions that reduce effectiveness, and the inclusion of non-bioactive or unstable compounds.

For example, many multi-vitamin formulations contain high concentrations of ingredients that may compete for absorption, leading to reduced efficacy. Additionally, herbal-based supplements often contain compounds with unknown synergistic effects, which can result in variable outcomes or unwanted side effects. Further, many current formulations lack targeted delivery mechanisms, resulting in poor patient adherence and inconsistent therapeutic benefits.

Accordingly, there remains a need for an improved health supplement formulation that provides optimized bioavailability, targeted ingredient interactions, and enhanced stability. Such a formulation should ensure compatibility between active ingredients, support efficient nutrient absorption, and maintain consistent therapeutic effects to maximize health benefits for users.

### SUMMARY

According to a first aspect of the disclosure, there is provided a health supplement composition comprising: a therapeutically effective amount of at least one active ingredient selected from Calcium Carbonate, Maize Starch, Magnesium Stearate, Microcrystalline Cellulose, Vitamin D3, and Vitamin K2; and at least one pharmaceutically acceptable excipient selected from the group consisting of binders, disintegrants, stabilizers, lubricants, diluents, and preservatives, wherein the composition is formulated for oral administration in a powder, tablet, capsule, or liquid dosage form.

In an embodiment, the composition is provided in a powder/granular form, typically packaged in tablets, as well as in a liquid form or as an additive to a liquid such as water, juice, or vegetable glycerine. Other delivery forms for the novel supplement may be utilized to meet particular operating circumstances or environments. Consequently, the disclosure is not limited to a specific delivery system but covers any and all suitable delivery systems.

The composition comprises essential vitamins and minerals carefully selected to maximize bioavailability, optimize ingredient synergy, and provide comprehensive nutritional support.

In an embodiment, the composition comprises Calcium Carbonate at 283 milligrams (mg), providing bone and cardiovascular health support. Maize Starch at 35 mg acts as a flow agent and energy support ingredient. Magnesium Stearate at 7 mg supports nervous system function and enzymatic activity. Microcrystalline Cellulose at 21 mg functions as a stabilizer and filler for structural integrity. Vitamin D3 at 375-500 micrograms (μg) supports bone health, immune function, and longevity. Vitamin K2 at 100 μg supports cardiovascular and bone health.

In another embodiment, Vitamin D3 can be provided in an amount of up to 3000 μg to specifically support male vitality and improve male erectile function.

Calcium, a primary component of the composition, plays a crucial role in bone formation, maintenance, and density, thereby reducing the risk of osteoporosis. It is also vital for blood clotting, nerve function, and the regulation of enzymes and hormones responsible for digestion, energy metabolism, and fat processing. The formulation utilizes Calcium Carbonate, Di-Calcium Phosphate, or Calcium Lactate to ensure effective calcium absorption and utilization.

Magnesium, an essential cofactor in over 300 biochemical reactions, contributes to normal muscle and nerve function, heart rhythm regulation, and immune support. It aids in blood sugar control, protein synthesis, and cardiovascular health. Magnesium Stearate is included in the composition to ensure optimal magnesium intake, preventing muscle cramps, metabolic dysfunction, and related deficiencies.

Vitamin D3 enhances calcium and phosphorus absorption, supporting strong bones and immune defense. It plays a regulatory role in gene expression linked to cancer prevention and autoimmune diseases. Additionally, Vitamin D3 may contribute to anti-aging properties by preserving telomere length, promoting cellular longevity, and reducing age-related decline.

Vitamin K2 is incorporated to facilitate calcium utilization, directing calcium to bones while preventing arterial calcification, thus enhancing cardiovascular health. It may also provide potential protective effects against cancer, particularly liver cancer, emphasizing its importance in maintaining long-term health.

Microcrystalline Cellulose serves as a stabilizer, emulsifier, and binding agent, ensuring tablet integrity and controlled ingredient release. This enhances formulation consistency, bioavailability, and ease of administration.

Maize Starch, a gluten-free ingredient, functions as an energy support and flow agent, contributing to the stability and homogeneity of the composition while enhancing energy metabolism.

By combining these essential nutrients in a scientifically optimized formulation, the present disclosure delivers a nutritionally balanced and highly bioavailable supplement.

In an embodiment, the composition is formulated to maximize synergistic interaction among the active ingredients to enhance bioavailability and therapeutic efficacy. The composition is further formulated to minimize potential ingredient interactions that could compromise stability, solubility, or bioavailability.

According to a second aspect of the disclosure, there is provided a method of enhancing vitality and immune support in a subject in need thereof, comprising administering to the subject a therapeutically effective amount of a health supplement composition, wherein the composition comprises: Calcium Carbonate, Maize Starch, Magnesium Stearate, Microcrystalline Cellulose, Vitamin D3, and Vitamin K2 and is formulated for oral administration in a powder, tablet, capsule, or liquid dosage form.

In an embodiment, the composition is administered once daily in a dosage sufficient to provide bone health support, cardiovascular health support, immune system enhancement, and overall metabolic function improvement, amongst others.

In an embodiment, the composition is administered once daily in a dosage sufficient to boost female vitality, support female reproductive function, enhance female libido, improve mood, and strengthen the immune system by providing advanced immune protection against diseases. Additionally, the composition supports a healthy nervous system, improves memory, provides cardiovascular support, and promotes optimal brain function. Furthermore, the composition may contribute to reduced cancer risk, offer reverse-aging properties, and provide anti-aging benefits, thereby supporting overall longevity and well-being.

In an embodiment, the composition is administered once daily in a dosage sufficient to boost male vitality, improve male erectile function, enhance male libido, improve male mood, and strengthen the immune system by providing advanced immune protection against diseases. Additionally, the composition supports a healthy nervous system, improves memory, provides cardiovascular support, and promotes optimal brain function. Furthermore, the composition may contribute to reduced cancer risk, offer reverse-aging properties, and provide anti-aging benefits, thereby supporting overall longevity and well-being.

The composition may be formulated to maximize synergistic interaction among the active ingredients to enhance bioavailability and therapeutic efficacy. The composition may be administered to a subject experiencing nutritional deficiencies, osteoporosis, immune suppression, or cardiovascular conditions.

In an embodiment, the health supplement composition is provided in a liquid form, comprising water or vegetable glycerine as a carrier medium, wherein the active ingredients include Calcium Lactate at 300 mg, Maize Starch at 35 mg, Magnesium Stearate at 7 mg, Microcrystalline Cellulose at 21 mg, Vitamin D3 at 375 μg, and Vitamin K2 at 100 μg. In another embodiment, the composition is provided in a powder form, comprising Calcium Carbonate at 283 mg, Maize Starch at 35 mg, Magnesium Stearate at 7 mg, Microcrystalline Cellulose at 21 mg, Vitamin D3 at 375 μg, and Vitamin K2 at 100 μg.

In an embodiment, the liquid formulation ensures homogeneous dispersion of the active ingredients, promoting efficient nutrient delivery and absorption while maintaining stability in suspension. The powder formulation is engineered for ease of administration in tablet, capsule, or dissolvable powder form, ensuring flexibility in dosage and user preference.

Both the liquid and powder formulations are developed to maximize synergistic interactions, enabling enhanced absorption, metabolic efficiency, and targeted therapeutic benefits. The formulations are further optimized to minimize undesired interactions, maintaining ingredient stability, solubility, and efficacy over time.

Thus, the present disclosure provides a nutritionally optimized, pharmaceutically stable, and therapeutically effective health supplement composition suitable for various administration routes, ensuring enhanced efficacy, stability, and bioavailability.

According to a further aspect of the disclosure, there is provided a method of manufacturing a health supplement composition, the method comprising one or more of the following steps: providing a therapeutically effective amount of at least one active ingredient selected from Calcium Carbonate, Maize Starch, Magnesium Stearate, Microcrystalline Cellulose, Vitamin D3, and Vitamin K2; mixing the active ingredients with at least one pharmaceutically acceptable excipient selected from the group consisting of binders, disintegrants, stabilizers, lubricants, diluents, and preservatives; formulating the mixture into an oral dosage form selected from tablets, capsules, powder, or liquid formulations; and packaging the composition in a pharmaceutically acceptable container for administration.

In an embodiment, the mixing step includes dry blending of the ingredients to ensure uniform distribution of the active components.

In an embodiment, the formulating step includes compression into tablets using a direct compression method.

In an embodiment, the formulating step includes encapsulation into hard gelatin capsules for controlled release.

In an embodiment, the formulating step includes dissolving the active ingredients in an aqueous or glycerine-based medium to produce a liquid formulation.

In an embodiment, the manufacturing process is conducted under Good Manufacturing Practices (GMP) conditions to ensure product stability, uniformity, and compliance with pharmaceutical standards.

In an embodiment, the final product is tested for stability, dissolution rate, and potency to ensure consistency in therapeutic efficacy.

In an embodiment, the final composition is packaged in blister packs, bottles, or single-dose sachets for optimal shelf stability and patient convenience.

These additions provide further depth to the health benefits of the individual ingredients, supporting the overall formulation's efficacy.

### BRIEF DESCRIPTION OF THE DRAWINGS

These and other features of this disclosure will become apparent from the following description of one example described with reference to the accompanying drawings in which:

FIG. 1 shows a health supplement composition, in accordance with an aspect of the disclosure; and

FIG. 2 shows a process of manufacturing a health supplement composition, in accordance with an aspect of the disclosure.

### DETAILED DESCRIPTION

The following description of the disclosure is provided as an enabling teaching of the disclosure. Those skilled in the relevant art will recognize that many changes can be made to the embodiment described, while still attaining the beneficial results of the present disclosure. It will also be apparent that some of the desired benefits of the present disclosure can be attained by selecting some of the features of the present disclosure without utilising other features. Accordingly, those skilled in the art will recognise that modifications and adaptions to the present disclosure are possible and can even be desirable in certain circumstances and are a part of the present disclosure. Thus, the following description is provided as illustrative of the principles of the present disclosure and limitation thereof. The components, relationships, and data flows shown are for example only; other variations may exist that employ similar principles.

Identical reference numerals are consistently used to indicate the same elements in all figures, unless a different numeral is explicitly assigned.

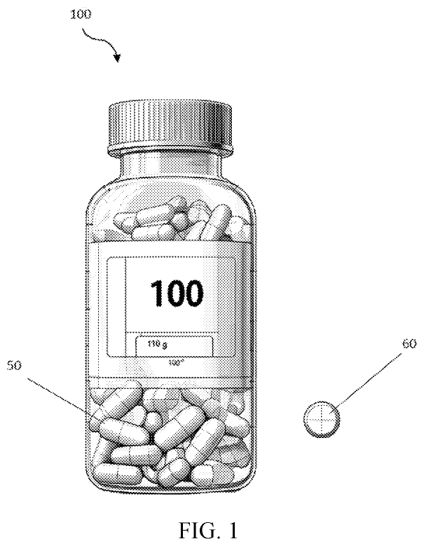

In FIG. 1, a health supplement composition, in accordance with aspects of the disclosure, is illustrated generally by numeral 100.

The health supplement composition 100 comprises a combination of active ingredients and pharmaceutically acceptable excipients formulated for oral administration in various dosage forms, including powder, tablet 60, capsule 50, or liquid form.

The active ingredients include: 283 mg of Calcium Carbonate, 35 mg of Maize Starch, 7 mg of Magnesium Stearate, 21 mg of Microcrystalline Cellulose, 375-500 μg of Vitamin D3, 100 μg of Vitamin K2.

The composition 100 is specifically designed to optimize bioavailability and promote synergistic interactions among its active ingredients. Calcium Carbonate serves as the primary calcium source, contributing to bone mineralization and skeletal integrity. Maize Starch functions as a binder and disintegrant, enhancing the dispersibility of the supplement for improved absorption. Magnesium Stearate is included as a lubricant to ensure smooth tablet compression and prevent caking, while also playing a role in enzymatic activity and neuromuscular function.

Microcrystalline Cellulose acts as a stabilizer and filler, maintaining the structural integrity of the dosage form and facilitating controlled release of active ingredients. Vitamin D3 supports calcium absorption and immune function, ensuring optimal bone health and metabolic regulation. Vitamin K2 plays a critical role in directing calcium deposition to bones and preventing vascular calcification, thereby enhancing cardiovascular health.

The formulation 100 is designed to maximize the synergistic effects of these ingredients, ensuring that each component contributes to the overall therapeutic efficacy of the supplement. By carefully selecting the proportions of each ingredient, the composition 100 minimizes competitive absorption effects and enhances nutrient bioavailability.

Additionally, the supplement 100 is formulated to be stable under standard storage conditions, maintaining its efficacy throughout its shelf life.

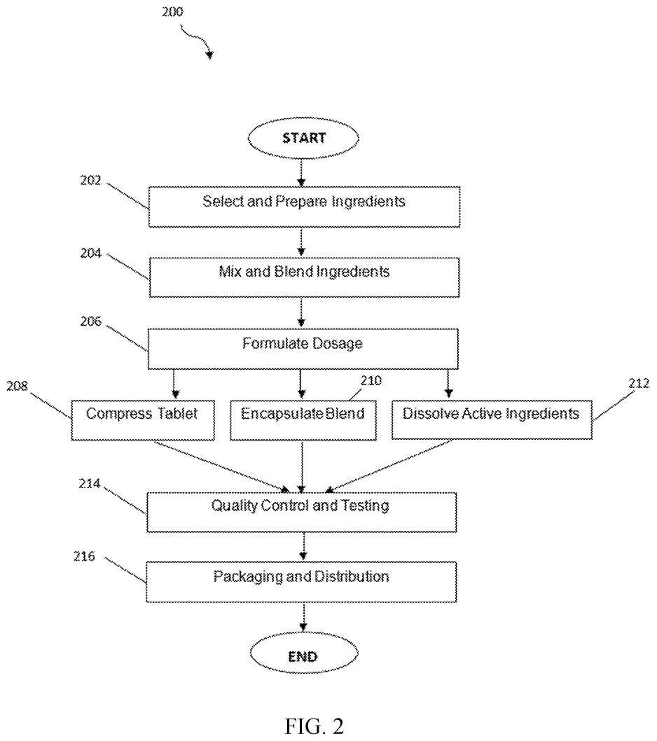

In FIG. 2, a process of manufacturing a health supplement composition, in accordance with aspects of the disclosure, is illustrated generally by numeral 200.

Referring to FIG. 2, the manufacturing process 200 comprises multiple stages designed to ensure uniformity, stability, and pharmaceutical integrity. The process begins with ingredient selection and preparation at block 202, where the active ingredients and excipients are sourced and tested for purity to confirm compliance with pharmaceutical standards.

Following this, at block 204, the selected ingredients undergo a mixing and blending process using dry blending techniques to achieve a homogeneous distribution of active and inactive components. This step is critical to maintaining consistency in the final dosage form.

At block 206, the blended mixture proceeds to the dosage formulation stage, where it is processed into the intended dosage format. Depending on the specific formulation, this stage includes:
(i) Tablet compression at block 208, where direct compression techniques are utilized to form tablets with consistent hardness, dissolution rates, and bioavailability.
(ii) Capsule encapsulation at block 210, where the mixture is filled into hard gelatin capsules to provide controlled release of the active ingredients.
(iii) Liquid formulation at block 212, where the active ingredients are dissolved in an aqueous or glycerine-based medium to create a liquid supplement for enhanced absorption and ease of administration.

Following dosage formulation, the product is subjected to rigorous quality control and testing at block 214. This stage includes stability assessments, dissolution rate analysis, and potency verification to ensure that the final composition maintains its therapeutic efficacy and meets pharmaceutical standards.

The final stage of the manufacturing process, at block 216, involves packaging and distribution. The composition is sealed in blister packs, bottles, or single-dose sachets to preserve stability, prevent contamination, and ensure ease of use for consumers.

The pharmaceutical composition of the present disclosure is produced using pharmaceutical techniques such as granulation, tablet compression, lyophilization, and sterile filtration. The disclosure is particularly well-suited for large-scale production, offering improved bioavailability, reduced dosage frequency, and enhanced patient compliance.

The composition can be prepared in various dosage forms, including tablets, capsules, suspensions, injectable solutions, or transdermal patches. Excipients may include binders such as microcrystalline cellulose and hydroxypropyl methylcellulose, disintegrants such as croscarmellose sodium and sodium starch glycolate, stabilizers such as antioxidants like ascorbic acid and tocopherols, chelating agents such as ethylenediaminetetraacetic acid (EDTA), solubility enhancers such as cyclodextrins and surfactants like Polysorbate 80, and controlled-release agents such as ethylcellulose, carnauba wax, and methacrylic acid copolymers.

The pharmaceutical composition is designed for oral, intravenous, or topical administration at a specific dosage per kilogram of body weight per day, ensuring a sustained plasma concentration of the active ingredient over a defined period. The formulation facilitates extended or modified release of the active ingredient, maintaining therapeutic levels while minimizing potential side effects.

In one embodiment, a tablet formulation is prepared by blending the active ingredient with excipients, followed by granulation using either a wet or dry granulation method before compression into tablets using a rotary press.

In another embodiment, the formulation undergoes accelerated stability testing at elevated temperatures and humidity over six months, demonstrating minimal degradation of the active ingredient and confirming improved stability compared to prior art formulations.

What is claimed is:

1. A composition comprising:
calcium carbonate in an amount of 150-400 milligrams (mg);
Vitamin D3 in an amount of 125 micrograms (μg) to 1,250 μg;
Vitamin K2 in an amount of 100-500 μg; and
at least one pharmaceutically acceptable excipient selected from the group consisting of binders, disintegrants, stabilizers, lubricants, diluents, and preservatives,
wherein the composition is formulated for oral administration in a powder, tablet, capsule, or liquid dosage form.

2. The composition of claim 1, wherein Vitamin D3 is present in an amount of 500-1,250 μg.

3. The composition of claim 1, wherein Vitamin K2 is present in an amount of 100-300 μg.

4. The composition of claim 1, wherein the composition is formulated for once-daily administration.

5. The composition of claim 1, wherein the composition comprises Vitamin D3 at about 1,250 μg and is formulated for once-weekly administration.

6. The composition of claim 1, wherein the composition is a liquid formulation and comprises distilled water as a carrier medium.

7. The composition of claim 1, wherein the composition is a liquid formulation and comprises vegetable glycerine as a carrier medium.

8. The composition of claim 1, wherein the composition is a liquid formulation and comprises distilled water and vegetable glycerine as carrier media.

9. A method of supporting bone mineralization and immune homeostasis in a subject, comprising:
administering to the subject a composition comprising:
calcium carbonate in an amount of 150-400 milligrams (mg);
Vitamin D3 in an amount of 125 micrograms (μg) to 1,250 μg;
Vitamin K2 in an amount of 100-500 μg; and
at least one pharmaceutically acceptable excipient selected from the group consisting of binders, disintegrants, stabilizers, lubricants, diluents, and preservatives,
wherein the composition is formulated for oral administration in a powder, tablet, capsule, or liquid dosage form.

10. The method of claim 9, wherein Vitamin D3 is present in an amount of 500-1,250 μg.

11. The method of claim 9, wherein Vitamin K2 is present in an amount of 100-300 μg.

12. The method of claim 9, wherein the composition is formulated for once-daily administration.

13. The method of claim 9, wherein the composition is formulated for once-weekly administration and comprises Vitamin D3 at about 1,250 μg.

14. The method of claim 9, wherein the composition is a liquid formulation and comprises distilled water as a carrier medium.

15. The method of claim 9, wherein the composition is a liquid formulation and comprises vegetable glycerine as a carrier medium.

16. The method of claim 9, wherein the composition is a liquid formulation and comprises distilled water and vegetable glycerine as carrier media.

17. A method of manufacturing a composition, comprising:
providing calcium carbonate in an amount of 150-400 milligrams (mg), Vitamin D3 in an amount of 125 micrograms (μg) to 1,250 μg, and Vitamin K2 in an amount of 100-500 μg;
mixing the calcium carbonate, Vitamin D3, and Vitamin K2 with at least one pharmaceutically acceptable excipient selected from the group consisting of binders, disintegrants, stabilizers, lubricants, diluents, and preservatives;
formulating the mixture into an oral dosage form selected from a tablet, capsule, powder, or liquid formulation; and
packaging the composition in a pharmaceutically acceptable container.

18. The method of claim 17, wherein the mixing step comprises dry blending the ingredients to achieve a homogeneous distribution of the active ingredients and excipients, and wherein the formulating step comprises compressing the blended mixture into tablets using a direct compression method.

19. The method of claim 17, wherein the formulating step comprises one of:
encapsulating the mixture into hard gelatin capsules for controlled release; or
dissolving the active ingredients in an aqueous or glycerine-based medium to produce the liquid formulation.

20. The method of claim 17, wherein the method is conducted under Good Manufacturing Practices (GMP) conditions, and wherein the packaged composition is tested for stability, dissolution rate, and potency prior to distribution.
